(19) United States
(12) Patent Application Publication
(10) Pub. No.: US 2020/0155863 A1
(43) Pub. Date: May 21, 2020

(54) METHOD AND DEVICE FOR MANAGING PACE-ASSISTED HIGH VOLTAGE DEFIBRILLATION SHOCKS

(71) Applicant: PACESETTER, INC., Sylmar, CA (US)

(72) Inventor: Xiaoyi Min, Simi Valley, CA (US)

(21) Appl. No.: 16/195,694

(22) Filed: Nov. 19, 2018

(51) Int. Cl. A61N 1/39

(52) U.S. Cl. CPC A61N 1/3956; A61N 1/3987; A61B 5/7275; A61N 1/3968; A61N 1/3925

(57) ABSTRACT

A method and system are provided. The method and system sense cardiac events of a heart. The method and system utilizes one or more processors. The processors detect a ventricular fibrillation (VF) episode based on the cardiac events and identify a pace-assisted VF therapy based on the ventricular fibrillation episode. The pace assisted VF therapy includes a burst pacing therapy and a high voltage (HV) shock. The method and system deliver the burst pacing therapy at one or more pacing sites in a coordinated manner before or during the HV shock. The one or more pacing sites includes at least one of a left ventricular (LV) site or a right ventricular (RV) site. The method and system deliver the HV shock along a shocking vector between shocking electrodes.

### BACKGROUND

Embodiments of the present disclosure generally relate to methods and devices for treating ventricular tachycardia (VT) arrhythmias and more particularly to methods and devices that

High voltage ventricular tachy therapies are delivered by an implantable cardioverter defibrillator (ICD) after the tachycardia episode is detected and classified.

Mapping of electrical activation and propagation has shown that high frequency (HF) pacing stimulus can capture the ventricular myocytes during VF and propagate. Also, it has been shown that high frequency pacing could capture the atrial myocytes and maintain regional control. HF pacing stimulus has been shown to be able to stimulate parasympathetic or sympathetic fibers during a refractory period. Altered autonomic tone is known to modulate cardiac arrhythmogenesis affect electrophysiological characteristics and ventricular fibrillation/defibrillation thresholds. A study has suggested that delivering high frequency pacing pulses prior to a shock with transvenous SVC-RV system improved the ventricular defibrillation thresholds.

However, the use of HF pacing pulses prior to HV shocks has exhibited limited success. A need remains for improved methods and systems for lowering ventricular defibrillation thresholds.

The HF was delivered through pacing electrode near the RV apex. However conceptually HF is preferably to be delivered to the low voltage gradient region during defibrillation shocks.

### SUMMARY

In accordance with embodiments herein, a method is provided. The method senses cardiac events of a heart. The method utilizes one or more processors. The processors declare a ventricular fibrillation (VF) episode based on the cardiac events and identifies a pace-assisted VF therapy based on the ventricular fibrillation episode. The pace assisted VF therapy includes a burst pacing therapy and a high voltage (HV) shock. The method delivers the burst pacing therapy at one or more pacing site in a coordinated manner before or during the HV shock. The one or more pacing site includes at least one of a left ventricular (LV) site or a right ventricular (RV) site. The method delivers the HV shock along a shocking vector between shocking electrodes.

Optionally, the burst pacing therapy may be delivered by at least one of multiple LV electrodes along an LV lead before the HV shock is delivered along at least one of the following shocking vectors: i) superior vena cava coil electrode and RV coil electrode to a CAN electrode, or ii) the RV coil electrode to the CAN electrode. The burst pacing therapy may be delivered by a leadless pacemaker in the RV before or during the HV shock is delivered along a shocking vector that may include a CAN electrode of a sub-cutaneous implantable medical device and a parasternal coil electrode. The burst pacing therapy may include a series of pacing pulses delivered at a frequency of at least 30 Hz and a pulse duration of 0.3-1.0 ms. The series of pacing pulses may be delivered for 0.5 seconds to 2 seconds at least one burst.

Optionally, the sensing of the cardiac events may include sensing the cardiac events at one or more RV sites or a set of at least four LV sites located proximate to a low voltage gradient region of the LV. The burst pacing therapy may be timed relative to the HV shock to cooperate with the HV shock to terminate fibrillation waves of the ventricular arrhythmia episode and to reduce a defibrillation threshold of the heart below a shock-only defibrillation threshold. The method may comprise collecting an electrical potential field distribution (EPFD) model of the heart; and may map electrode positions into EPFD model. The EPFD model may be scaled based on measurements of electrical potentials at the RV and LV sites while applying a known voltage to an HV shocking electrode.

Optionally, the burst pacing therapy may be delivered to at least one of an LV region or RV region that may exhibit a voltage gradient characteristic of interest. The burst pacing therapy may be delivered before or during the HV shock, and the HV shock may have a voltage of at least 200V. The HV shock may include a group of multiple low-voltage HV shocks at voltage level in a range of 50V to 100 V, the group of low-voltage HV shocks may include at least three shocks.

In accordance with embodiments herein, an implantable medical system is provided. The system comprises electrodes that are configured to sense cardiac events. At least a portion of the electrodes are configured to be located proximate to a pacing site at one of a left ventricular (LV) site or a right ventricular (RV) site. At least a portion of the electrodes are configured to define a shocking vector. An implantable medical device (IMD) comprises memory and one or more processors. The memory stores program instructions. The one or more processors that, when executing the program instructions, are configured to declare a ventricular fibrillation (VF) episode based on the cardiac events and identify a pace-assisted VF therapy based on the ventricular fibrillation episode. The pace assisted VF therapy includes a burst pacing therapy and a high voltage (HV) shock. The system delivers the burst pacing therapy at one or more pacing sites in a coordinated manner before or during the HV shock. The one or more pacing site includes at least one of a left ventricular (LV) site or a right ventricular (RV) site. The system delivers the HV shock along the shocking vector.

Optionally, the system may comprise an LV lead coupled to a housing of the IMD. The LV lead may have multiple LV electrodes. At least one of the LV electrodes may be configured to be located proximate to the LV site corresponding to the pacing site and may deliver the burst pacing therapy. A second lead may have at least one of a superior vena cava (SVC) coil electrode or an RV coil electrode. The shock vector may include a CAN of the IMD and at least one of the SVC coil electrode or the RV coil electrode. The IMD may represent a subcutaneous implantable cardioverter defibrillators (S-ICD). The system may further comprise a leadless pacemaker in communication with the S-ICD. The S-ICD may be positioned in a mid-axillary position and may be coupled to parasternal lead having a parasternal coil electrode.

Optionally, the one or more processors may be configured to analyze a timing of VF beats to obtain at least one of a VF cycle length (CL) or variation and may determine at least one of a number of pulses in a pulse train of high frequency pacing therapy for each VF beat based on at least one of the VF cycle length or variation. The one or more processors may be configured to set a timing delay to time the HF pacing therapy such that one or more of pulses therefrom occur during a period of time in which a local tissue region surrounding the pacing site is excitable and not refractory.

Optionally, the one or more processors may be configured to set a frequency of the burst pacing therapy at a high frequency relative to a cycle length of non-fibrillation arrhythmias. The memory may be configured to store a model of a voltage gradient experienced across a heart during a HV shock only defibrillation stimulation. The burst pacing therapy may be delivered before or during the HV shock is delivered. The one or more processors may be further configured to collect an electrical potential field distribution (EPFD) model of the heart, map electrode positions into EPFD model, scale the EPFD model based on measurements of electrical potentials at the LV sites while applying a known voltage to an HV shocking electrode.

### BRIEF DESCRIPTION OF THE DRAWINGS

FIG. 1 illustrates an IMD and external device coupled to a heart in a patient and implemented in accordance with embodiments herein.

FIG. 2A shows a block diagram of an exemplary IMD that is configured to be implanted into the patient in accordance with embodiments herein.

FIG. 2B illustrates a graphical representation of an implantable medical system that is configured to apply pacer assisted VF therapy in accordance with embodiments herein.

FIG. 3A illustrates a process for coordinating a pacer assisted VF therapy that includes a burst pacing therapy and high-voltage shock in accordance with embodiments herein.

FIG. 3B illustrates a model of a voltage gradient experienced across a heart during a HV shock only defibrillation stimulation in accordance with embodiments herein.

FIG. 4 illustrates a process for modeling an electrical field (EF) map of a heart in accordance with embodiments herein.

FIG. 5 illustrates a process for defining a pace-assisted VF therapy in accordance with embodiments herein.

FIG. 6 illustrates a functional block diagram of the external device in accordance with embodiments herein.

FIG. 7 illustrates a distributed processing system in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods and systems described herein may employ all or portions of structures or aspects of various embodiments discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, where indicated, the methods may be fully or partially implemented by one or more processors of one or more devices or systems. While the operations of some methods may be described as performed by the processor(s) of one device, additionally, some or all of such operations may be performed by the processor(s) of another device described herein.

The term “obtain” or “obtaining”, as used in connection with data, signals, information and the like, includes at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The term “pace-assisted defibrillation threshold” refers to a minimum amount of energy needed to be delivered in an HV shock, delivered in combination with a secondary or supporting paced stimulation, in order to return a heart to a normal rhythm from a condition in which the heart is experiencing a fibrillation dysrhythmia episode.

The term “shock-only defibrillation threshold” refers to a minimum amount of energy needed to be delivered in an HV shock, alone without any secondary or supporting electrical stimulation, in order to return a heart to a normal rhythm from a condition in which the heart is experiencing a fibrillation dysrhythmia episode.

The terms “high-voltage shock” and “HV shock” refer to defibrillation stimulus delivered at an energy level sufficient to terminate a defibrillation episode in a heart, wherein the energy level is defined in Joules to be at least 10 J, and more preferably 15 J-50 J and/or the energy level is defined in terms of voltage to be at least 200 V, preferably to be at least 600 V and more preferably between 600V and 900 V.

The terms “high frequency” and “HF”, as used in connection with pacing pulses and pacing therapy refer to delivering pacing pulses at a rate greater than a rate associated with anti-tachycardia pacing, namely at a rate of at least 30 Hz.

Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference. Additionally or alternatively, the IMD may include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method Including The Same”, which are hereby incorporated by reference. Additionally or alternatively, the IMD may include one or more structural and/or functional aspects of the device(s) described in U.S. Pat. No. 8,391,980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the IMD may include one or more structural and/or functional aspects of the device(s) described in U.S. application Ser. No. 15/973,195, titled “Subcutaneous Implantation Medical Device With Multiple Parastemal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties.

Further, one or more combinations of IMDs may be utilized from the above incorporated patents and applications in accordance with embodiments herein.

### Implantable Medical Device

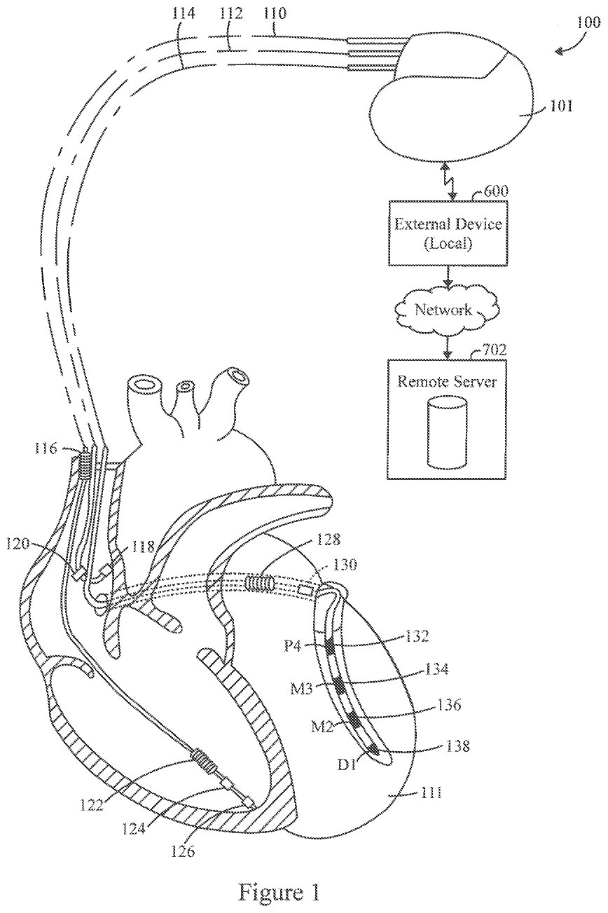

FIG. 1 illustrates an IMD 100 and external device 600 coupled to a heart 111 in a patient and implemented in accordance with one embodiment. The external device 600 may be a programmer, an external defibrillator, a workstation, a portable computer, a personal digital assistant, a cell phone, a bedside monitor and the like. The IMD may represent a cardiac monitoring device, pacemaker, cardioverter, cardiac rhythm management device, defibrillator, neurostimulator, leadless monitoring device, leadless pacemaker and the like, implemented in accordance with one embodiment of the present invention. The IMD 100 may be a dual-chamber stimulation device capable of treating both fast and slow arrhythmias with stimulation therapy, including cardioversion, defibrillation, anti-tachycardia pacing and pacing stimulation, as well as capable of detecting heart failure, evaluating its severity, tracking the progression thereof, and controlling the delivery of therapy and warnings in response thereto. The IMD 100 may be controlled to sense atrial and ventricular waveforms of interest, discriminate between two or more ventricular waveforms of interest, deliver stimulus pulses or shocks, and inhibit application of a stimulation pulse to a heart based on the discrimination between the waveforms of interest and the like. Exemplary structures for the IMD 100 are discussed and illustrated in the drawings herewith.

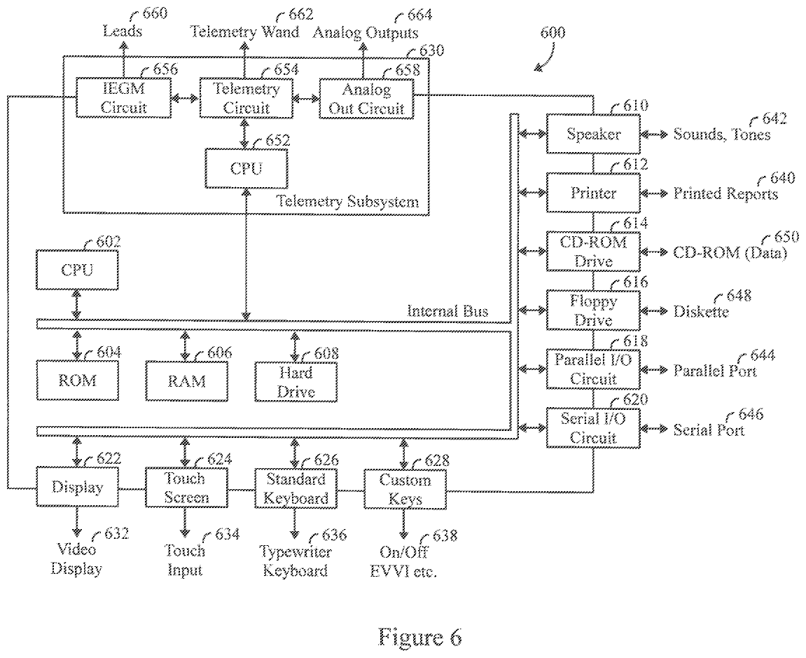

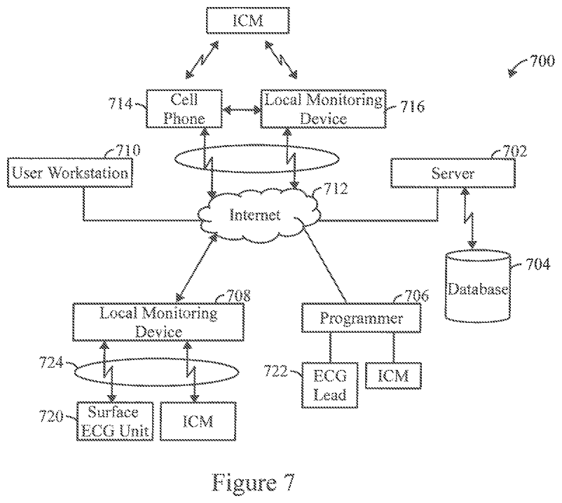

The IMD 100 communicates with a local external device 600 discussed hereafter in connection with FIG. 6. The local external device 600 communicates with a remote server 702 discussed hereafter in connection with FIG. 7.

The IMD 100 includes a housing 101 that is joined to a header assembly that holds receptacle connectors connected to a right ventricular lead 110, a right atrial lead 112, and a coronary sinus lead 114, respectively. The leads 112, 114 and 110 measure cardiac signals of the heart 111. The right atrial lead 112 includes an atrial tip electrode 118 and an atrial ring electrode 120. The coronary sinus lead 114 includes a left atrial ring electrode 128, a left atrial coil electrode 130 and one or more left ventricular electrodes 132-138 (e.g., also referred to as P1, M1, M2 and D1) to form a multi-pole LV electrode combination. The right ventricular lead 110 includes an RV tip electrode 126, an RV ring electrode 124, an RV coil electrode 122, and an SVC coil electrode 116. The leads 112, 114 and 110 detect IEGM signals that are processed and analyzed as described herein. The leads 112, 114 and 110 also delivery therapies as described herein.

During implantation, the external device 600 is connected to one or more of the leads 112, 114 and 110 through temporary inputs. The inputs of the external device 600 receive IEGM signals from the leads 112, 114 and 110 during implantation and display the IEGM signals to the physician on a display. Optionally, the external device 600 may not be directly connected to the leads 112, 114 and 110. Instead, the IEGM cardiac signals sensed by the leads 112, 114 and 110 may be collected by the IMD 100 and then transmitted wirelessly to the external device 600. Hence, the external device 600 receives the IEGM cardiac signals through telemetry circuit inputs. The physician or another user controls operation of the external device 600 through a user interface.

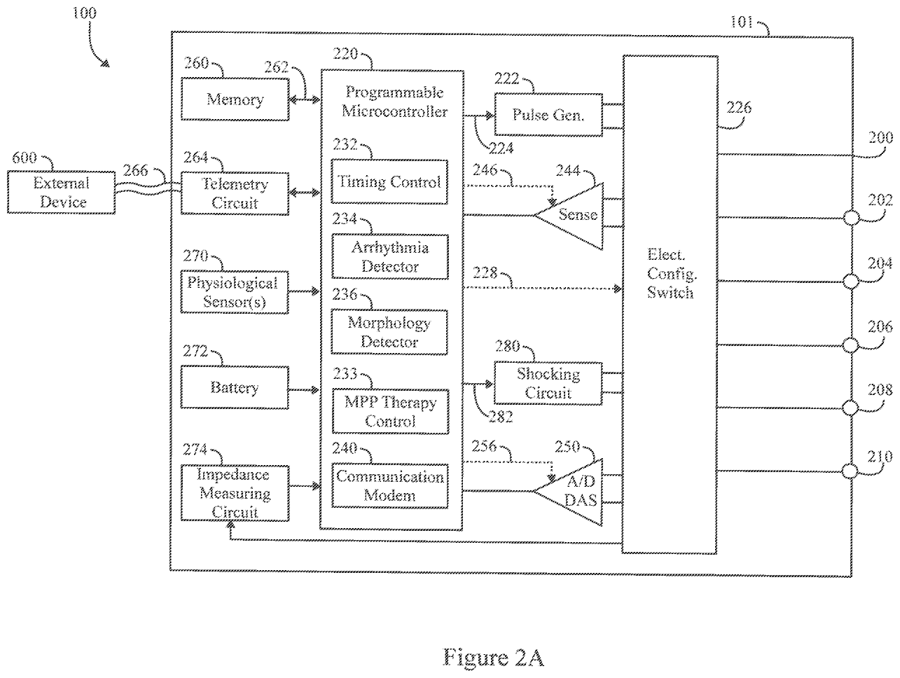

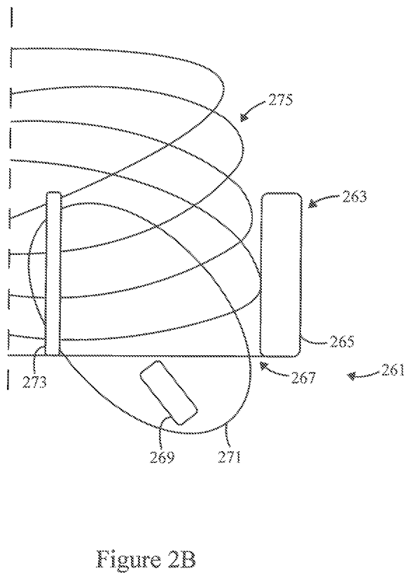

FIG. 2A shows a block diagram of an exemplary IMD 100 that is configured to be implanted into the patient. The IMD 100 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 100 has a housing 101 to hold the electronic/computing components. The housing 101 (which is often referred to as the “can,” “case,” “encasing,” or “case electrode”) may be programmably selected to act as the return electrode for certain stimulus modes. The housing 101 further includes a connector (not shown) with a plurality of terminals 200-210. The terminals may be connected to electrodes that are located in various locations within and about the heart. The type and location of each electrode may vary. For example, the electrodes may include various combinations of ring, tip, coil, shocking electrodes, and the like.

The IMD 100 includes a programmable microcontroller 220 that controls various operations of the IMD 100, including cardiac monitoring and stimulation therapy. The microcontroller 220 includes a microprocessor (or equivalent control circuitry), one or more processors, RAM and/or ROM memory, logic and timing circuitry, state machine circuitry, and I/O circuitry. The IMD 100 further includes a ventricular pulse generator 222 that generates stimulation pulses for connecting the desired electrodes to the appropriate I/O circuits, thereby facilitating electrode programmability. The switch 226 is controlled by a control signal 228 from the microcontroller 220.

A pulse generator 222 is illustrated in FIG. 2. Optionally, the IMD 100 may include multiple pulse generators, similar to the pulse generator 222, where each pulse generator is coupled to one or more electrodes and controlled by the microcontroller 220 to deliver select stimulus pulse(s) to the corresponding one or more electrodes. The IMD 100 includes sensing circuit 244 selectively coupled to one or more electrodes that perform sensing operations, through the switch 226 to detect the presence of cardiac activity in the chamber of the heart 111. The output of the sensing circuit 244 is connected to the microcontroller 220 which, in turn, triggers, or inhibits the pulse generator 222 in response to the absence or presence of cardiac activity. The sensing circuit 244 receives a control signal 246 from the microcontroller 220 for purposes of controlling the gain, threshold, polarization charge removal circuitry (not shown), and the timing of any blocking circuitry (not shown) coupled to the inputs of the sensing circuit 224.

In the example of FIG. 2, the sensing circuit 244 is illustrated. Optionally, the IMD 100 may include multiple sensing circuits 244, where each sensing circuit is coupled to one or more electrodes and controlled by the microcontroller 220 to sense electrical activity detected at the corresponding one or more electrodes. The sensing circuit 224 may operate in a unipolar sensing configuration or a bipolar sensing configuration.

The IMD 100 further includes an analog-to-digital (A/D) data acquisition system (DAS) 250 coupled to one or more electrodes via the switch 226 to sample cardiac signals across any pair of desired electrodes. The A/D converter 250 is configured to acquire intracardiac electrogram signals, convert the raw analog data into digital data and store the digital data for later processing and/or telemetric transmission to an external device 600 (e.g., a programmer, local transceiver, or a diagnostic system analyzer). The A/D converter 250 is controlled by a control signal 256 from the microcontroller 220.

The switch 226 is managed, by the microcontroller 220, to sense the cardiac events at an LV electrode along an LV lead that includes multiple LV electrodes and to enable the burst pacing therapy to be delivered by at least one of the multiple LV electrodes before the HV shock is delivered along at least one of the following shocking vectors: i) superior vena cava coil electrode and RV coil electrode to a CAN electrode, or ii) the RV coil electrode to the CAN electrode.

The switch 226 may be coupled to an LV lead having multiple LV electrodes, at least one of the LV electrodes configured to be located proximate to the LV site corresponding to the pacing site and to deliver the burst pacing therapy. The switch 226 may be further coupled to a second lead with at least one of a superior vena cava (SVC) coil electrode or an RV coil electrode, the shock vector including a CAN of the IMD and at least one of the SVC coil electrode or the RV coil electrode.

The microcontroller 220 is operably coupled to a memory 260 by a suitable data/address bus 262. The programmable operating parameters used by the microcontroller 220 are stored in the memory 260 and used to customize the operation of the IMD 100 to suit the needs of a particular patient. The operating parameters of the IMD 100 may be non-invasively programmed into the memory 260 through a telemetry circuit 264 in telemetric communication via communication link 266 (e.g., MICS, Bluetooth low energy, and/or the like) with the external device 600.

The IMD 100 can further include one or more physiological sensors 270. Such sensors are commonly referred to as “rate-responsive” sensors because they are typically used to adjust pacing stimulation rates according to the exercise state of the patient. However, the physiological sensor 270 may further be used to detect changes in cardiac output, changes in the physiological condition of the heart, or diurnal changes in activity (e.g., detecting sleep and wake states). Signals generated by the physiological sensors 270 are passed to the microcontroller 220 for analysis. While shown as being included within the IMD 100, the physiological sensor(s) 270 may be external to the IMD 100, yet still, be implanted within or carried by the patient. Examples of physiological sensors might include sensors that, for example, sense respiration rate, pH of blood, ventricular gradient, activity, position/posture, minute ventilation (MV), and/or the like.

A battery 272 provides operating power to all of the components in the IMD 100. The battery 272 is capable of operating at low current drains for long periods of time, and is capable of providing a high-current pulses (for capacitor charging) when the patient requires a shock pulse (e.g., in excess of 2 A, at voltages above 2 V, for periods of 10 seconds or more). The battery 272 also desirably has a predictable discharge characteristic so that elective replacement time can be detected. As one example, the IMD 100 employs lithium/silver vanadium oxide batteries.

The IMD 100 further includes an impedance measuring circuit 274, which can be used for many things, including sensing respiration phase. The impedance measuring circuit 274 is coupled to the switch 226 so that any desired electrode and/or terminal may be used to measure impedance in connection with monitoring respiration phase. The IMD 100 is further equipped with a communication modem (modulator/demodulator) 240 to enable wireless communication with other devices, implanted devices and/or external devices. In one implementation, the communication modem 240 may use high frequency modulation of a signal transmitted between a pair of electrodes. As one example, the signals may be transmitted in a high frequency range of approximately 10-80 kHz, as such signals travel through the body tissue and fluids without stimulating the heart or being felt by the patient.

The microcontroller 220 further controls a shocking circuit 280 by way of a timing control 232. The shocking circuit 280 generates shocking pulses of low (e.g., up to 0.5 joules), moderate (e.g., 0.5-10 joules), or high energy (e.g., 10 to 40 joules), as controlled by the microcontroller 220. The shocking circuit 280 is controlled by the microcontroller 220 by a control signal 282.

Although not shown, the microcontroller 220 may further include other dedicated circuitry and/or firmware/software components that assist in monitoring various conditions of the patient's heart and managing pacing therapies. The microcontroller 220 further includes a timing control 232, an arrhythmia detector 234, a morphology detector 236 and pace assisted VF therapy controller 233. The timing control 232 is used to control various timing parameters, such as stimulation pulses (e.g., pacing rate, atria-ventricular (AV) delay, atrial interconduction (A-A) delay, ventricular interconduction (V-V) delay, etc.) as well as to keep track of the timing of RR-intervals, refractory periods, blanking intervals, noise detection windows, evoked response windows, alert intervals, marker channel timing, and the like. The timing control 232 controls a timing for delivering the burst pacing therapy in a coordinated manner before or during the HV shock. The timing control 232 controls the burst pacing therapy timed relative to the HV shock to cooperate with the HV shock to terminate fibrillation waves of the ventricular arrhythmia episode and to reduce a defibrillation threshold of the heart below a shock-only defibrillation threshold.

The morphology detector 236 is configured to review and analyze one or more features of the morphology of CA signals. For example, in accordance with embodiments herein, the morphology detector 236 may analyze the morphology of detected R waves, where such morphology is then utilized to determine whether to include or exclude one or more beats from further analysis. For example, the morphology detector 236 may be utilized to identify non-conducted ventricular events, such as ventricular fibrillation and the like.

The arrhythmia detector 234 is configured to apply one or more arrhythmia detection algorithms for detecting arrhythmia conditions. By way of example, the arrhythmia detector 234 may apply various VF detection algorithms. The arrhythmia detector 234 is configured to declare a ventricular fibrillation (VF) episode based on the cardiac events.

The therapy controller 233 is configured to perform the operations described herein. The therapy controller 233 is configured to identify a pace-assisted VF therapy based on the ventricular fibrillation episode, the pace assisted VF therapy including a burst pacing therapy and a high voltage (HV) shock. The therapy controller 233 is configured to manage delivery of the burst pacing therapy at a pacing site in a coordinated manner before or during the HV shock. The pacing site is located at one of a left ventricular (LV) site or a right ventricular (RV) site. The therapy controller 233 is configured to manage delivery of the HV shock along a shocking vector between shocking electrodes. The therapy controller 233 is further configured to collect an electrical potential field distribution (EPFD) model of the heart; and map electrode positions into EPFD model. The therapy controller 233 is further configured to scale the EPFD model based on measurements of electrical potentials at the RV and/or LV sites (by the sensing circuit 244) while the microcontroller 220 directs the shocking circuit 280 to apply a known voltage to an HV shocking electrode.

The therapy controller 233 is further configured to deliver the HV shock as a group of multiple low-voltage HV shocks at a voltage level in a range of 50V to 100 V, the group of low-voltage HV shocks including at least three shocks. The therapy controller 233 is further configured to analyze a timing of VF beats to obtain at least one of a VF cycle length (CL) or variation and to determine at least one of a number of pulses in a pulse train of the burst pacing therapy or a duration of pulse train of the burst pacing therapy based on at least one of the VF cycle length or variation. The therapy controller 233 may be further configured to set a timing delay to time the burst pacing therapy such that one or more of pulses therefrom occur during a period of time in which a local tissue region surrounding the pacing site is excitable and not refractory. The therapy controller 233 may be configured to set a frequency of the burst pacing therapy at a high frequency relative to a cycle length of non-fibrillation arrhythmias.

The memory 260 may be configured to store a model of a voltage gradient experienced across a heart during a HV shock only defibrillation stimulation. The therapy controller 233 may be further configured to collect an electrical potential field distribution (EPFD) model of the heart; map electrode positions into EPFD model; and scale the EPFD model based on measurements of electrical potentials at the RV and/or LV sites.

In accordance with embodiments, the IMD 100 may represent a subcutaneous implantable cardioverter defibrillators (S-ICD). The communication modem 240 is configured to wirelessly communicate with a leadless pacemaker, such as to pass timing information there between. The communication modem 240 may transmit timing information to a leadless pacemaker such as when sending an instruction for the leadless pacemaker to deliver a burst pacing therapy. The communication modem 240 may receive timing information from a leadless pacemaker such as when receiving a direction from the leadless pacemaker that a burst pacing therapy has been delivered or is currently being delivered and that S-ICD should now deliver the HV shock.

### Subcutaneous Implantable Medical Device

FIG. 2B illustrates a graphical representation of an implantable medical system 261 that is configured to apply pacer assisted VF therapy in accordance with embodiments herein. The system 261 includes a subcutaneous implantable medical device (SIMD) 263 that is configured to be implanted in a subcutaneous area exterior to the heart. The SIMD 263 is positioned in a subcutaneous area or region, and more particularly in a mid-axillary position along a portion of the rib cage 275. The system 261 also includes a leadless pacemaker 269 implanted within the heart, such as at an apex 271 of the right ventricle. The system 261 does not require insertion of a transvenous lead.

The pulse generator 265 may be implanted subcutaneously and at least a portion of the lead 267 may be implanted subcutaneously. In particular embodiments, the SIMD 263 is an entirely or fully subcutaneous SIMD. Optionally, the SIMD 263 may be positioned in a different subcutaneous region.

The SIMD 263 includes a pulse generator 265 and at least one lead 20 that is operably coupled to the pulse generator 265. The “at least one lead” is hereinafter referred to as “the lead.” Nevertheless, it should be understood that the term, “the lead,” may mean only a single lead or may mean more than one single lead. The lead 267 includes at least one electrode segment 273 that is used for providing high-voltage shocks for defibrillation. Optionally, the lead 267 may include one or more sensing electrodes. The pulse generator 265 includes a housing that forms or constitutes an electrode utilized to deliver HV shocks. The electrode associated with the housing of the pulse generator 265 is referred to as the “CAN” electrode.

In an alternative embodiment, the lead 267 may include one or more electrode segments, in which the electrode segments are spaced apart from one another having an electrical gap therebetween. The lead body may extend between the gap. One electrode segment may be positioned along an anterior of the chest, while another electrode segment may be positioned along a lateral and/or posterior region of the patient. The electrode segments may be portions of the same lead, or the electrode segments may be portions of different leads. The electrode segments may be positioned subcutaneously at a level that aligns with the heart of the patient for providing a sufficient amount of energy for defibrillation. The lead 20010 includes a lead body that extends from the mix-auxiliary position along an inter-costal area between ribs and oriented with the coil electrode(s) extending along the sternum (e.g., over the sternum or parasternally within one to three centimeters from the sternum). A proximal end the coil electrodes may be located proximate to the xiphoid process.

### Coordination of LV Pacing and HV Shock Therapy

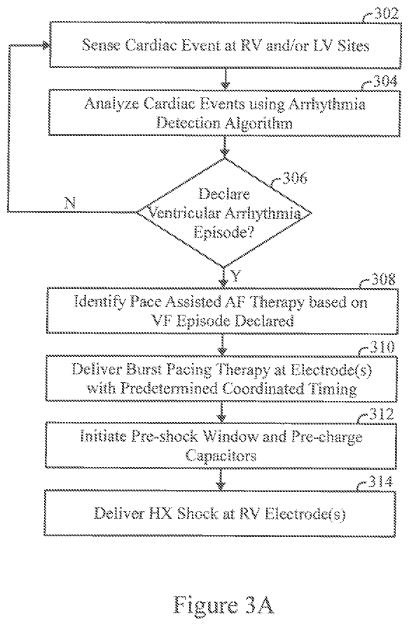

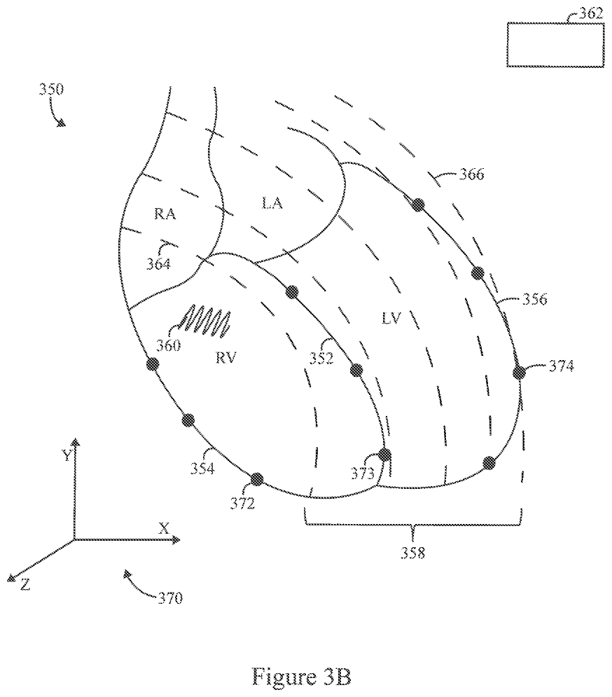

FIG. 3A illustrates a process for coordinating a pacer assisted VF therapy that includes a burst pacing therapy and high-voltage shock in accordance with embodiments herein. The process of FIG. 3A may be implemented by one or more processors within or distributed between the IMD, a local external device and/or a remote server.

At 302, cardiac events are sensed by electrodes located proximate to one or more right ventricular (RV) sites and one or more left ventricular (LV) sites of the heart. The cardiac events are sensed over a period of time corresponding to a detection period. At 304, the one or more processors analyze the cardiac events utilizing various arrhythmia detection algorithms, such as algorithms that the detect bradycardia, fibrillation and/or tachycardia events.

At 306, the one or more processors determine whether to declare a ventricular fibrillation (VF) episode based on the analysis of the cardiac events. When a VF episode is not declared, flow returns to 302. Alternatively, when a VF episode is declared, flow continues to 308.

At 308, the one or more processors identify a pace assisted VF therapy to be delivered. The pace assisted VF therapy is identified based on the nature and/or characteristics of the ventricular arrhythmia episode that was declared. As explained herein, the pace assisted VF therapy includes a burst pacing therapy and one or more high voltage shocks to be delivered in a coordinated manner, such as based on a predetermined timing relation there between. The predetermined timing relation between the burst pacing therapy and the HV shocks is defined to change a condition of the heart to have a pace assisted defibrillation threshold, which is lower than the shock only defibrillation threshold of the heart that would otherwise be exhibited, but for the burst pacing therapy.

For example, the predetermined timing relation may be that the entire burst pacing therapy is delivered before the defibrillation HV shocks. Alternatively, the predetermined timing relation may be that the burst pacing therapy is started before the defibrillation HV shock, but continues and in temporal overlapping manner with the defibrillation HV shock. Additionally or alternatively, the predetermined timing relation may be that the entire burst pacing therapy is delivered in a temporal overlapping manner during the defibrillation HV shocks.

The identification of the pace assisted VF therapy includes setting one or more therapy related parameters including one or more pacing related parameters and one or more HV shock related parameters. Non-limiting examples of the pacing related parameters include the electrode combination to deliver the pacing pulses, a number of pacing pulses to be delivered during a pacing burst or pulse train, pulse width, pulse amplitude, pulse frequency (e.g., also referred to as the pulse to pulse or interpulse delay) and the like.

For example, the pacing related parameters may define the burst pacing to have a high frequency, such as at least 30 Hz, or preferably a frequency between 50 and 100 Hz, or more preferably at 50 Hz. Additionally or alternatively, the pacing related parameters may be defined in terms of the pulse to pulse (Interpulse) separation between successive pacing pulses, such as no more than 30 ms, and as another example between 15 and 25 ms, and more preferably 20 ms. The pacing related parameters may define the pulse duration, such as to be 0.3 to 1.0 ms, or 0.4 to 0.8 ms, or more preferably 0.5 ms, by way of example, the burst pacing therapy may include one or more burst, where each burst delivers the pacing pulses for 0.5 seconds to 2 seconds. The pacing related parameters may define the pacing pulses to be unipolar or bipolar. In at least certain embodiments, it may be preferable to use unipolar pacing pulses. As one particular configuration, the burst parameters may be a 50 Hz high frequency pacing pulses (20 ms inter-pulse separation), and with unipolar stimulation having pulse durations of 0.5 ms.

The pacing related parameters may designate a combination of LV electrodes to simultaneously deliver the pacing pulses. The combination of LV electrodes may be designated to correspond to an LV region that exhibits a predetermined voltage gradient characteristic. The characteristic of the voltage gradient may represent a lowest voltage gradient. For example, one more LV electrodes may be selected, for delivering the pacing pulses, that are located proximate to heart tissue region exhibiting a low or lowest voltage gradient characteristic. The lowest voltage gradient may be determined relative to voltage gradients of other regions of the heart. For example, the heart may be segmented into discrete voltage gradient regions or a continuous voltage gradient. The voltage gradients for the various regions may be compared to determine the region or portion of the continuous voltage gradient that is lowest with respect to other discrete voltage gradient regions or other portions of the continuous voltage gradient. As another example, the LV electrodes may be selected for any region of the LV that exhibits a voltage gradient below a voltage gradient threshold. As a further example, when an LV lead includes four electrodes, the pacing related parameters may designate a combination of one or more of the LV electrodes to deliver the burst pacing therapy (e.g., P1, M2, M3 and D1 electrodes simultaneously deliver each pacing pulse). Alternatively, a subset of the LV electrodes may be utilized to deliver the burst pacing therapy. Additionally or alternatively, the burst pacing therapy may include delivery of separate pacing pulse bursts at separate combinations of LV electrodes. For example, first and second pacing pulse bursts may be delivered during different time intervals, or during partially overlapping time intervals, to corresponding first and second separate combinations of LV electrodes. As one example, the first pacing pulse burst may be delivered between the P4 and M3 electrodes, while the second pacing pulse burst may be delivered between the M2 and D1 electrodes, where starting times of the first and second pacing pulse burst are staggered by a predetermined number of milliseconds. As a further example, the burst pacing therapy may define multiple pacing vectors between one or more RV electrodes, or between the CAN electrode and various combinations of LV electrodes. For example, the burst pacing therapy may define first, second and third pacing pulse bursts to be delivered between corresponding first, second and third CAN-LV pacing vectors (e.g., CAN-D1, CAN-M2, CAN-M3, CAN-P4).

Non-limiting examples of HV shock related parameters include the electrode combination that delivers the HV shock, the polarity designation between cathode and anode electrodes within the electrode combination, shock voltage, pulse width, a number of phases within the shock (e.g., monophasic, biphasic, tri-phasic) and the like.

While the operation for identifying the pace assisted AF therapy is illustrated at 308 in the process of FIG. 3A, it is understood that the pace assisted AF therapy may be identified at various other points in time. For example, the pace assisted AF therapy may be identified before, during or after the sensing of cardiac events at 302. Optionally, the pace assisted AF therapy may be identified before, during or after the analysis of the cardiac events at 304, before, during or after the declaration at 306. Optionally, the pace assisted AF therapy may be identified at a point in time entirely separate from the operations of FIG. 3A.

At 310, the one or more processors deliver a burst pacing therapy at one or more electrodes with a predetermined coordinated timing relative to an HV shock. The location at which the burst pacing therapy is delivered is dependent upon the overall electrode configuration. For example, in accordance with embodiments herein, when an MPP lead is located along the LV, the burst pacing therapy is delivered at one or more LV electrodes. Additionally or alternatively, when an S-ICD is used in combination with a leadless pacemaker in the RV, the burst pacing therapy is delivered at one or more RV locations. For example, the predetermined coordinated timing may time a beginning and/or end of the burst pacing therapy to occur before or during a start time of the HV shock. Additionally or alternatively, the burst pacing therapy may be delivered at both one or more RV sites and one or more LV sites. At 312, the one or more processors initiate a pre-shock window and begin pre-charging the capacitors used to deliver the HV shock. Additionally, the one or more processors monitor a pace assisted coordination interval in connection with the predetermined coordinated timing. At 314, the one or more processors deliver a high voltage (HV) shock at one or more RV electrodes.

The pace assisted coordination interval coordinates delivery of the burst pacing therapy at a time relative to the HV shock, such that the pacing pulses cooperate with the HV shock to terminate fibrillation waves of a ventricular fibrillation episode in a more efficient manner, as compared to delivering an HV shock alone. Stated another way, the pace assisted coordination interval times delivery of the burst pacing therapy to reduce a defibrillation threshold of the surrounding tissue (also referred to as a pace-assisted defibrillation threshold) as compared to a shock only defibrillation threshold that would otherwise be necessary to achieve defibrillation when delivering an HV shock alone (also referred to as a shock-only defibrillation threshold). The burst pacing therapy effectively places the tissue in a state susceptible to a pace-assisted defibrillation threshold that is lower than the shock-only defibrillation threshold otherwise associated with the tissue.

Additionally or alternatively, the predetermined coordinated timing may define the high-frequency burst pacing therapy to be delivered after the defibrillation HV shocks. Additionally or alternatively, the predetermined coordinated timing may define the high-frequency burst pacing therapy to be delivered at a time during delivery of the defibrillation HV shocks such that the pacing pulses are timed as a fibrillation wave propagates from the shocking site in order to capture excitable gaps by utilizing an LV lead or a leadless pacer.

The timing delay at 312 and frequency of the pulses within the burst seek to time the burst pacing therapy such that one or more of the pulses occur during a period of time in which a local tissue region surrounding the pacing site is excitable and not refractory. The frequency of the burst pacing therapy is set at a high frequency relative to cycle lengths of non-fibrillation arrhythmias. For example, during ventricular tachycardia (VT), each VT event progresses through any local region of the heart at with a cycle length that is dependent on the VT rate. For example, a VT rate of 200 beats per minute (bpm) would exhibit a cycle length between successive beats of 3.33 Hz (e.g., 3.33 beats per second). In a general sense, a local tissue region would at least partially transition between an excitable state and a refractory state 3.33 times per second. A VT rate of 300 beats per minute would correspond a cycle length of 2.0 Hz, and thus local tissue regions would transition between excitable and refractory states 2 times per second.

In accordance with embodiments herein, the frequency of the burst pacing is set substantially higher than a frequency associated with VT or other non-fibrillation arrhythmias. Thus, one or more of the pulses in the burst pacing therapy will be delivered between successive points in time when the local tissue region transitions to the excitable state due to the VF episode. Consequently, at least one of the pulses from the burst pacing therapy will capture the local tissue region surrounding the pacing site and cause the local tissue region to enter a refractory state “out of cycle” with the fibrillation waveform. Disrupting the fibrillation waveforms in regions of the heart that experience low voltage gradients from HV shocks has the effect of lowering the defibrillation threshold.

FIG. 3B illustrates a model of a voltage gradient experienced across a heart during a HV shock only defibrillation stimulation. In FIG. 3B, a heart 350 is illustrated with a right atrial (RA) chamber, right ventricular (RV) chamber, left atrial (LA) chamber, and left ventricular (LV) chamber. The heart 350 also includes a septum wall 352, RV anterior wall 354 and LV posterior wall 356. The heart 350 will experience different voltage gradients depending on various factors such as the physiologic health of the heart, the present state of the heart 350 within a cardiac cycle or arrhythmia, HV shock defibrillation vectors and the like.

In the example of FIG. 3B, a shocking vector is defined between an RV coil 360 and a CAN electrode 362. The shocking vector has an associated gradient field 358 illustrated by a series of dashed lines that generally represent progressively decreasing electrical potential and field distribution experienced by the heart. For example, the region along gradient field line 364 would experience a higher voltage gradient as compared to the region along gradient field line 366. The gradient field lines in between lines 364 and 366 would experience progressively decreasing voltage potentials at increased distance from the RV coil 360.

By way of example, in a transvenous system, an RV lead is placed in the right ventricle and an LV lead is placed along the left ventricle, while an optional SVC lead may be placed in the SVC. For example, the LV lead may include four electrodes. The four electrodes are located in or near a low voltage gradient region of interest for which the LV tissue will experience a low voltage gradient during delivery of a defibrillation HV shock from the transvenous RV lead. In accordance with embodiments herein, burst pacing pulses are delivered prior to or during delivery of the defibrillation HV shock through one or multiple electrodes along the LV to help terminate defibrillation waves in combination with the HV shock in a more efficient manner. Additionally or alternatively, the LV lead may deliver bipolar pacing pulses and/or different combinations of pacing pulses from different combinations of the LV leads.

As another example, embodiments may be implemented in connection with subcutaneous implantable cardioverter defibrillators (S-ICD) operating in combination with a lead-based or leadless pacemaker. For example, the S-ICD may be positioned with a mid-axillary CAN and a parasternal coil electrode. In this configuration, the apex of the heart would represent the region of the heart that would experience a lower voltage gradient as compared to other areas of the heart closer to the parasternal coil or CAN electrodes. The leadless pacemaker (or a tip of a pacemaker lead) is positioned at the RV apex or septum. The leadless pacemaker (or tip of the pacemaker lead) would deliver a high frequency (HF) pacing therapy in order to reduce the defibrillation threshold to a pace-assisted defibrillation threshold as compared to a shock-only defibrillation threshold that would otherwise be experienced by the S-ICD system.

In the foregoing example, a single defibrillation HV shock was described as part of the pace-assisted VF therapy. Additionally or alternatively, the pace-assisted VF therapy may include a group of multiple low-voltage defibrillation shocks. The low-voltage defibrillation shocks may be at voltages of less than 100 V and more preferably be in the range of 50 to 100 V. The group of low-voltage defibrillation shocks may include more than three shocks, but less than 10 shocks, and more preferably between 5 and 7 low-voltage defibrillation shocks. The low-voltage defibrillation shocks may be delivered along the same defibrillation vector as utilized in connection with high-voltage defibrillation shocks. Non-limiting examples of defibrillation vectors include SVC coil to RV coil, and/or combining the SVC coil and CAN of the IMD at a common polarity, while the RV coil is assigned an opposite polarity.

While the low-voltage shocks are delivered, the burst pacing therapy may be delivered simultaneously to the region of the heart exhibiting a low voltage gradient. For example, in a transvenous system that includes an RV lead and an SVC lead, the defibrillation vectors may be between the CAN of the IMD and the RV coil electrode and/or between the CAN of the IMD, the SVC coil and the RV coil. In the foregoing configuration, an LV lead having one or more LV electrodes may be utilized to deliver the burst pacing therapy.

Alternatively, in a configuration that utilizes a subcutaneous ICD and a leadless pacer, the subcutaneous ICD may deliver the multiple low-voltage shocks between one or more defibrillation vectors between the CAN of the ICD and the subcutaneous electrodes. Simultaneously, the leadless pacer would deliver the burst pacing therapy to the apex of the RV and/or other locations in the RV where the leadless pacer is implanted.

Conceptually, the heart may be viewed as a collection of reentrant circuits that carry ventricular fibrillation waves. In accordance with embodiments herein, the pace-assisted VF therapy affords a greater chance of capturing ventricular fibrillation waves as such fibrillation waves propagate through different regions of the heart, thereby terminating an associated number of reentrant circuits. For example, when the defibrillation electrode configuration defines 2 defibrillation vectors and the pacing electrode configuration defines one pacing vector, the configuration affords the opportunity to capture ventricular fibrillation waves propagating along three or more separate reentrant circuits/regions. Additionally or alternatively, when 2 defibrillation vectors and two or more pacing vectors are utilized, the combined configuration affords the opportunity to capture fibrillation waves propagating along 4 or more reentrant circuits, such as 5 reentrant circuits.

### Electrical Field Modeling

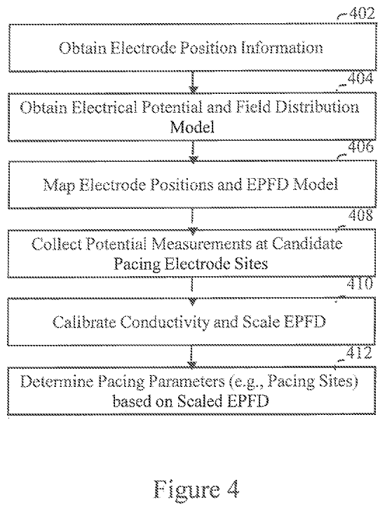

FIG. 4 illustrates a process for modeling an electrical field (EF) map of a heart in accordance with embodiments herein. The process of FIG. 4 may be implemented before implant of an IMD, during implantation of an IMD or after implant of an IMD. The process of FIG. 4 may be performed in connection with an individual patient such that the EF map is specific to a particular patient. Additionally or alternatively, the process may be performed in connection a population of patients such that one or more EF maps are generated as general models. Different models may be associated with different types of patients (e.g., based on sex, gender, age, weight, ethnic background) and/or physiologic characteristics of the heart (e.g., heart conditions, past history of arrhythmias). The process may be periodically or automatically repeated to check or update an EF map for an individual patient such as to account for changes in a patient's heart condition over time.

At 402, one or more processors obtain electrode position information for electrodes positioned in or proximate to the heart. For example, when RV and LV leads are implanted in the heart, the electrode position information may designate one or more RV sites and one or more LV sites for corresponding RV and LV electrodes. The electrode position information may be defined in various manners. As one example, each electrode position may be defined as a location vector (distance and angle) with respect to particular anatomical features of the heart (e.g., with respect to the RV apex, LV apex, septum wall, right atrial appendage, left atrial appendage, left anterior descending artery, mitral valve, tricuspid valve). Additionally or alternatively, the electrode positions may be defined as a set of coordinates within a reference coordinate system. The reference coordinate system may be defined in connection with a model of the heart. For example, anatomical features of the heart may also be defined as a set of coordinates within the reference coordinate system.

The electrode position information may be determined through the use of medical diagnostic imaging, such as through 3-D x-ray examinations taken after or in connection with implanting the RV and LV leads and IMD. Optionally, alternative systems may be utilized to determine the electrode positions, such as ultrasound imaging, 3-D visualization and navigation systems (e.g., the MediGuide® system) and the like.

At 404, the one or more processors obtain a general or candidate electrical potential and field distribution (EPFD) model. The EPFD model comprises a collection of electrical potentials and/or field values, each of which is assigned to a point in the EPFD model (e.g., assigned to a coordinate location and/or anatomical location). The general EPFD model may be defined based on a patient population and/or based on prior measurements from a current patient. The general EPFD model enables changes/gradients to be determined for the electrical potential and field distribution across the regions of the heart (e.g., the septum wall, LV wall).

With reference to FIG. 3B, the EPFD model may be defined relative to a coordinate system 370 (e.g., Cartesian or Polar coordinate system) with EP points 372-374 throughout the coordinate system 370 defining corresponding values for the local electrical potential.

At 406, the one or more processors map the electrode positions onto the EPFD model. For example, electrode designators (e.g., RV coil, RV tip, LV-P1, LV-M2, LV-M3, LV-D4) may be assigned to points (e.g., coordinates or anatomical features) in the EPFD model. Each electrode designator would then be associated with a corresponding electrical potential and/or field for the point in the EPFD model. While not shown in FIG. 3B, electrode designators could be assigned at coordinates within the coordinate system 370.

At 408, the one or more processors collect electrical potential measurements at one or more of the electrodes. For example, the processors may apply a known voltage between the HV shocking electrodes, and measure the electrical potential (e.g., current and/or voltage) at one or more LV electrodes.

At 410, the one or more processors calibrate conductivity within the heart and scale the electrical fields of the EPFD model based on the measured potentials. For example, with reference to FIG. 3B, the processors adjust the EP values associated with the coordinate points 372-374 within the EPFD model from a standard or expected field distribution to a patient specific field distribution. The calibration and scaling accounts for the circumstance when the actual potential measured at the coordinate points differs from the standard or expected value. Accordingly, the EPFD model is adjusted to be patient specific and more reflective of a particular heart conductivity.

At 412, the one or more processors determine one or more pacing parameters based on the patient specific scaled EPFD model. For example, the processors may determine a subset of one or more of the LV electrodes to be utilized in connection with delivering a burst pacing therapy. Additionally or alternatively, the processors may designate one or more of the LV electrodes to be a cathode, while a CAN of the IMD is designated as the anode in connection with delivering burst pacing therapy that includes unipolar pacing pulses. As another example, the processors may designate two or more of the LV electrodes as cathodes, while the CAN of the IMD remains the anode in connection with delivering burst pacing therapy that includes bipolar pacing pulses.

Optionally, the designation of unipolar or bipolar pacing therapy and the designation of which LV electrodes to be utilized may be preprogrammed and determined without use of the process of FIG. 4.

Optionally, the process of FIG. 4 may be modified to provide an additional or alternative mapping approach. For example, in addition to or as an alternative, electrode positions in the blood chamber or relative to cardiac anatomy may be obtained. A patient specific EPFD model may be built for the blood chamber or cardiac anatomy of the present patient based on electrical potential measurements collected at the candidate pacing electrode sites. The pacing parameters maybe then be determined based on the patient specific EPFD model.

### Defining Pace Assisted VF Therapy

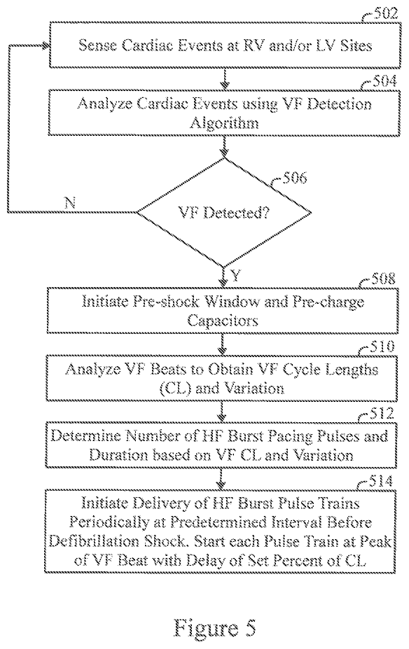

FIG. 5 illustrates a process for defining a pace-assisted VF therapy in accordance with an alternative embodiment. At 502, cardiac events are sensed by electrodes located proximate to one or more right ventricular (RV) sites and one or more left ventricular (LV) sites of the heart. The cardiac events are sensed over a period of time corresponding to a detection period. At 504, the one or more processors analyze the cardiac events utilizing a fibrillation detection algorithm. At 506, the one or more processors determine whether to declare a ventricular fibrillation (VF) episode based on the analysis of the cardiac events. When a VF episode is not declared, flow returns to 502. Alternatively, when a VF episode is declared, flow continues to 508.

At 508, the one or more processors initiate a pre-shock window and begin pre-charging the capacitors during which additional cardiac events are collected for a desired amount of time or for a desired number of VF beats. At 510, the one or more processors analyze a timing of VF beats to obtain at least one of a VF cycle length (CL) or variation and to determine at least one of a number of pulses in a pulse train of high frequency pacing therapy for each VF beat based on at least one of the VF cycle length or variation. For example, the VF cycle length may correspond to an average or mean of VF cycle lengths between the VF beats in the series, with a variation corresponding to a deviation from the average or mean.

At 512, the one or more processors determine a number of HF pacing pulses and a duration of the HF to be delivered as the burst pacing therapy. The determination at 512 is based on, among other things, the VF cycle length and variation. At 514, the one or more processors initiate delivery of an HF pacing therapy. The HF pacing therapy includes a series or train of pacing pulses that are delivered with a predetermined pulse to pulse interval. By way of example, the pulse to pulse interval within a single pulse train may be defined as a select percentage of the VF cycle length. For example, the HF pacing therapy may comprise delivery of first and second pulse trains, wherein the pulse to pulse interval between the pulses within the first pulse train is set to be approximately 80% of the VF cycle length. Additionally or alternatively, the pulse to pulse interval of the second pulse train may be set based on a similar percentage of the VF cycle length and/or a different percentage of the VF cycle length.

The HF pacing therapy also defines an interval between successive trains of pacing pulses. For example, the interval between successive trains of pacing pulses may be timed to start each pulse train at a peak of a VF beat. The start point of each train of pacing pulses may be initiated based on a direct measurement of a peak of a VF beat. Alternatively, the start point of each train of pacing pulses may be initiated based on an estimation of a peak of each VF beat, where the estimation may be derived from, among other things, the measurements of VF cycle length and variation.

The ventricular waves travel through the heart tissue at a fibrillation wavelength and cycle length that may be constant or vary. Accordingly, it is difficult to time delivery of a particular pacing pulse at a pacing site to fall within the portion of the fibrillation cycle length in which the local tissue is excitable, as opposed to in a refractory state. The process of FIG. 5 seeks to time pulses of the burst pacing therapy such that one or more of the pulses occur during a period of time which a local tissue region surrounding the pacing site is excitable and not refractory.

### External Device

FIG. 6 illustrates a functional block diagram of the external device 600 that is operated in accordance with the processes described herein and to interface with implantable medical devices as described herein. The external device 600 may be a workstation, a portable computer, an IMD programmer, a PDA, a cell phone and the like. The external device 600 includes an internal bus that connects/interfaces with a Central Processing Unit (CPU) 602, ROM 604, RAM 606, a hard drive 608, the speaker 610, a printer 612, a CD-ROM drive 614, a floppy drive 616, a parallel I/O circuit 618, a serial I/O circuit 620, the display 622, a touch screen 624, a standard keyboard connection 626, custom keys 628, and a telemetry subsystem 630. The internal bus is an address/data bus that transfers information between the various components described herein. The hard drive 608 may store operational programs as well as data, such as waveform templates and detection thresholds.

The CPU 602 typically includes a microprocessor, a micro-controller, or equivalent control circuitry, designed specifically to control interfacing with the external device 600 and with the IMD 100. The CPU 602 performs the COI measurement process discussed above. The CPU 602 may include RAM or ROM memory, logic and timing circuitry, state machine circuitry, and I/O circuitry to interface with the IMD 100. The display 622 (e.g., may be connected to the video display 632). The touch screen 624 may display graphic information relating to the IMD 100. The display 622 displays various information related to the processes described herein. The touch screen 624 accepts a user's touch input 634 when selections are made. The keyboard 626 (e.g., a typewriter keyboard 636) allows the user to enter data to the displayed fields, as well as interface with the telemetry subsystem 630. Furthermore, custom keys 628 turn on/off 638 (e.g., EWI) the external device 600. The printer 612 prints copies of reports 640 for a physician to review or to be placed in a patient file, and speaker 610 provides an audible warning (e.g., sounds and tones 642) to the user. The parallel I/O circuit 618 interfaces with a parallel port 644. The serial I/O circuit 620 interfaces with a serial port 646. The floppy drive 616 accepts diskettes 648. Optionally, the floppy drive 616 may include a USB port or other interface capable of communicating with a USB device such as a memory stick. The CD-ROM drive 614 accepts CD ROMs 650.

The telemetry subsystem 630 includes a central processing unit (CPU) 652 in electrical communication with a telemetry circuit 654, which communicates with both an IEGM circuit 656 and an analog out circuit 658. The circuit 656 may be connected to leads 660. The circuit 656 is also connected to the implantable leads 660 to receive and process IEGM cardiac signals as discussed above. Optionally, the IEGM cardiac signals sensed by the leads 660 may be collected by the IMD 100 and then transmitted, to the external device 600, wirelessly to the telemetry subsystem 630 input.

The telemetry circuit 654 is connected to a telemetry wand 662. The analog out circuit 658 includes communication circuits to communicate with analog outputs 664. The external device 600 may wirelessly communicate with the IMD 100 and utilize protocols, such as Bluetooth, GSM, infrared wireless LANs, HIPERLAN, 3G, satellite, as well as circuit and packet data protocols, and the like. Alternatively, a hard-wired connection may be used to connect the external device 600 to the IMD 100.

### Distributed Processing System (Server Based)

FIG. 7 illustrates a distributed processing system 700 in accordance with one embodiment. The distributed processing system 700 includes a remote server 702 connected to a database 704, a programmer 706, a local RF transceiver 708 and a user workstation 710 electrically connected to a communication system 712. Any of the processor-based components in FIG. 7 (e.g., workstation 710, cell phone 714, PDA 716, server 702, programmer 706, IMD 100) may perform the processes discussed above.

The communication system 712 may be the internet, a voice over IP (VoIP) gateway, a local plain old telephone service (POTS) such as a public switched telephone network (PSTN), a cellular phone based network, and the like. Alternatively, the communication system 712 may be a local area network (LAN), a campus area network (CAN), a metropolitan area network (MAN), or a wide area network (WAM). The communication system 712 serves to provide a network that facilitates the transfer/receipt of information such as cardiac signal waveforms, ventricular and atrial heart rates.

The server 702 is a computer system that provides services to other computing systems over a computer network. The server 702 controls the communication of information such as cardiac signal waveforms, ventricular and atrial heart rates, and detection thresholds. The server 702 interfaces with the communication system 712 to transfer information between the programmer 706, the local RF transceiver 708, the user workstation 710 as well as a cell phone 714 and a personal data assistant (PDA) 716 to the database 704 for storage/retrieval of records of information. On the other hand, the server 702 may upload raw cardiac signals from an implanted lead 722, surface ECG lead 722 or the IMD 100 via the local RF transceiver 708 or the programmer 706.

The database 704 stores information such as cardiac signal waveforms, ventricular and atrial heart rates, thresholds, and the like, for a single or multiple patients. The information is downloaded into the database 704 via the server 702 or, alternatively, the information is uploaded to the server from the database 704. The programmer 706 is similar to the external device 600 and may reside in a patient's home, a hospital, or a physician's office. The programmer 706 interfaces with the lead 722 and the IMD 100. The programmer 706 may wirelessly communicate with the IMD 100 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 706 to the IMD 100. The programmer 706 is able to acquire cardiac signals from the surface of a person (e.g., ECGs), intra-cardiac electrogram (e.g., IEGM) signals from the IMD 100, and/or cardiac signal waveforms, ventricular and atrial heart rates, and detection thresholds from the IMD 100. The programmer 706 interfaces with the communication system 712, either via the internet or via POTS, to upload the information acquired from the surface ECG unit 720, the lead 722 or the IMD 100 to the server 702.

The local RF transceiver 708 interfaces with the communication system 712 to upload one or more of cardiac signal waveforms, ventricular and atrial heart rates, and detection thresholds to the server 702. In one embodiment, the surface ECG unit 720 and the IMD 100 have a bi-directional connection 724 with the local RF transceiver 708 via a wireless connection. The local RF transceiver 708 is able to acquire cardiac signals from the surface of a person, intra-cardiac electrogram signals from the IMD 100, and/or cardiac signal waveforms, ventricular and atrial heart rates, and detection thresholds from the IMD 100. On the other hand, the local RF transceiver 708 may download stored cardiac signal waveforms, ventricular and atrial heart rates, and detection thresholds, and the like, from the database 704 to the surface ECG unit 720 or the IMD 100.

The user workstation 710 may interface with the communication system 712 via the Internet or POTS to download cardiac signal waveforms, ventricular and atrial heart rates, and detection thresholds via the server 702 from the database 704. Alternatively, the user workstation 710 may download raw data from the surface ECG unit 720, lead 722 or IMD via either the programmer 706 or the local RF transceiver 708. Once the user workstation 710 has downloaded the cardiac signal waveforms, ventricular and atrial heart rates, or detection thresholds, the user workstation 710 may process the information in accordance with one or more of the operations described above. The user workstation 710 may download the information and notifications to the cell phone 714, the PDA 716, the local RF transceiver 708, the programmer 706, or to the server 702 to be stored on the database 704. For example, the user workstation 710 may communicate data to the cell phone 714 or PDA 716 via a wireless communication link.

### CLOSING STATEMENTS

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A method, comprising:
sensing cardiac events of a heart;
utilizing one or more processors to perform:
detecting a ventricular fibrillation (VF) episode based on the cardiac events;
identifying a pace-assisted VF therapy based on the ventricular fibrillation episode, the pace assisted VF therapy including a burst pacing therapy and a high voltage (HV) shock;
delivering the burst pacing therapy at one or more pacing site in a coordinated manner before or during the HV shock, the one or more pacing site including at least one of a left ventricular (LV) site or a right ventricular (RV) site; and
delivering the HV shock along a shocking vector between shocking electrodes.

2. The method of claim 1, wherein the burst pacing therapy is delivered by at least one of multiple LV electrodes along an LV lead before the HV shock is delivered along at least one of the following shocking vectors: i) superior vena cava coil electrode and RV coil electrode to a CAN electrode, or ii) the RV coil electrode to the CAN electrode.

3. The method of claim 1, wherein the burst pacing therapy is delivered by a leadless pacemaker in the RV before or during the HV shock is delivered along a shocking vector that includes a CAN electrode of a sub-cutaneous implantable medical device and a parasternal coil electrode.

4. The method of claim 1, wherein the burst pacing therapy includes a series of pacing pulses delivered at a frequency of at least 30 Hz and a pulse duration of 0.3-1.0 ms, the series of pacing pulses delivered for 0.5 seconds to 2 seconds at least one burst.

5. The method of claim 1, wherein the sensing of the cardiac events includes sensing the cardiac events at one or more RV sites or a set of at least four LV sites located proximate to a low voltage gradient region of the LV.

6. The method of claim 1, wherein the burst pacing therapy is timed relative to the HV shock to cooperate with the HV shock to terminate fibrillation waves of the ventricular arrhythmia episode and to reduce a defibrillation threshold of the heart below a shock-only defibrillation threshold.

7. The method of claim 1, further comprising collecting an electrical potential field distribution (EPFD) model of the heart; and mapping electrode positions into EPFD model.

8. The method of claim 7, wherein the EPFD model is scaled based on measurements of electrical potentials at the RV and LV sites while applying a known voltage to an HV shocking electrode.

9. The method of claim 1, wherein the burst pacing therapy is delivered to at least one of an LV region or RV region that exhibits a voltage gradient characteristic of interest.

10. The method of claim 1, wherein the burst pacing therapy is delivered before or during the HV shock, and the HV shock has a voltage of at least 200V.

11. The method of claim 1, wherein the HV shock includes a group of multiple low-voltage HV shocks at voltage level in a range of 50V to 100 V, the group of low-voltage HV shocks including at least three shocks.

12. An implantable medical system, comprising:
a plurality of electrodes, at least a portion of the plurality of electrodes configured to sense cardiac events, at least a portion of the electrodes configured to be located proximate to a pacing site at one of a left ventricular (LV) site or a right ventricular (RV) site, at least a portion of the electrodes configured to define a shocking vector;
an implantable medical device (IMD) that comprises memory and one or more processors, the memory to store program instructions; and the one or more processors that, when executing the program instructions, are configured to:
detect a ventricular fibrillation (VF) episode based on the cardiac events;
identify a pace-assisted VF therapy based on the ventricular fibrillation episode, the pace assisted VF therapy including a burst pacing therapy and a high voltage (HV) shock;
deliver the burst pacing therapy at one or more pacing sites in a coordinated manner before or during the HV shock, the one or more pacing site including at least one of a left ventricular (LV) site or a right ventricular (RV) site; and
deliver the HV shock along the shocking vector.

13. The system of claim 12, further comprising an LV lead coupled to a housing of the IMD, the LV lead having multiple LV electrodes, at least one of the LV electrodes configured to be located proximate to the LV site corresponding to the pacing site and to deliver the burst pacing therapy; and a second lead with at least one of a superior vena cava (SVC) coil electrode or an RV coil electrode, the shock vector including a CAN of the IMD and at least one of the SVC coil electrode or the RV coil electrode.

14. The system of claim 12, wherein the IMD represents a subcutaneous implantable cardioverter defibrillators (S-ICD), the system further comprising a leadless pacemaker in communication with the S-ICD, wherein the S-ICD is positioned in a mid-axillary position and is coupled to parasternal lead having a parasternal coil electrode.

15. The system of claim 12, wherein the one or more processors are configured to analyze a timing of VF beats to obtain at least one of a VF cycle length (CL) or variation and to determine at least one of a number of pulses in a pulse train of high frequency pacing therapy for each VF beat based on at least one of the VF cycle length or variation.

16. The system of claim 12 wherein the one or more processors are configured to set a timing delay to time the HF pacing therapy such that one or more of pulses therefrom occur during a period of time in which a local tissue region surrounding the pacing site is excitable and not refractory.

17. The system of claim 12, wherein the one or more processors are configured to set a frequency of the burst pacing therapy at a high frequency relative to a cycle length of non-fibrillation arrhythmias.

18. The device of claim 12, wherein the memory is configured to store a model of a voltage gradient experienced across a heart during a HV shock only defibrillation stimulation.

19. The system of claim 12, wherein the burst pacing therapy is delivered before or during the HV shock is delivered.

20. The system of claim 12, wherein the one or more processors is further configured to collect an electrical potential field distribution (EPFD) model of the heart; map electrode positions into EPFD model; scale the EPFD model based on measurements of electrical potentials at the LV sites while applying a known voltage to an HV shocking electrode.
